(19) United States
(12) Patent Application Publication
(10) Pub. No.: US 2004/0005634 A1
(43) Pub. Date: Jan. 8, 2004

(54) System and method for determining differential protein expression, diagnostic biomarker discovery system and method of using the same, and protein biomarkers and therapeutic and diagnostic uses thereof

(76) Inventors: Edward F. Patz JR., Chapel Hill, NC (US); Michael J. Campa, Durham, NC (US); Michael C. Fitzgerald, Durham, NC (US)

Correspondence Address: FLESHNER & KIM, LLP, P.O. BOX 221200, CHANTILLY, VA, 20153, US

(21) Appl. No.: 10/402,138

(22) Filed: Mar. 31, 2003

Related U.S. Application Data
(63) Continuation-in-part of application No. 09/902,786, filed on Jul. 12, 2001.

(51) Int. Cl. G01N 33/53; G06F 19/00; G01N 33/48; G01N 33/50; C12M 1/34

(52) U.S. Cl. 435/7.1; 702/19; 435/287.2

(57) ABSTRACT

The present invention relates generally to systems and methods for determining differential protein expression, and diagnostic discovery systems and methods that utilize the same. In particular, the present invention relates to a system and method of obtaining and analyzing protein profiles to determine protein patterns associated with clinical parameters and manifestations of disease and to discover specific biomarkers that are characteristic of diseases such as cancer. The present invention further relates to the identification of potential molecular targets for diagnostic applications and/or therapeutic intervention.

### BACKGROUND OF THE INVENTION

1. Field of the Invention

The present invention relates generally to systems and methods for determining differential protein expression, and diagnostic discovery systems and methods that utilize the same. In particular, the present invention relates to a system and method of obtaining and analyzing protein profiles to determine protein patterns associated with clinical parameters and manifestations of disease and to discover specific biomarkers that are characteristic of diseases such as cancer. The present invention further relates to the identification of potential molecular targets for diagnostic applications and/or therapeutic intervention.

2. Background of the Related Art

There is a continuing need for innovative strategies that allow early detection, diagnosis, treatment, monitoring and prognosis of diseases, such as cancer and other biological conditions, and inability to tolerate certain medications or treatments. While current non-invasive radiologic studies and laboratory tests play an integral role in the evaluation of diseases and biological conditions, there are clear limitations for early detection and specific diagnosis. For example, early detection efforts and screening trials for various cancers, even targeted at high risk individuals, have often been ineffectual. See, for example: Fontana, R. S. et al., “Early Lung Cancer Detection: Results of the Initial (Prevalence) Radiologic and Cytologic Screening in the Mayo Clinic Study”, Am. Rev. Respir. Dis. 130: 561-565 (1984); Berlin, N. I., et al., “The National Cancer Institute Cooperative Early Lung Cancer Detection Program: Results of the Initial Screen (Prevalence)”, Am. Rep. Respir. Dis. 130: 545-549 (1984); Kubik, A. and Polak, J., “Lung Cancer Detection: Results of a Randomized Prospective Study in Czechoslovakia”, Cancer 57: 2427-2437 (1986); Fontana, R. S. et al., “The Mayo Lung Project for Early Detection and Localization of Bronchogenic Carcinoma: A Status Report”, Chest 67: 511-522 (1975); Tockman, M. S., “Survival and Mortality from Lung Cancer in a Screened Population. The Johns Hopkins Study”, Chest 89 (suppl.): 324S-325S (1986); Fontana, R. S. et al., “Screening for Lung Cancer. A Critique of the Mayo Lung Project”, Cancer 67: 1,155-1,164 (1991); and Marcus, P. M. et al., “Lung Cancer Mortality in the Mayo Lung Project: Impact of Extended Follow-up”, J. Natl. Cancer Inst. 92: 1,308-1,315 (2000). Thus an alternative approach to early detection, accurate diagnosis and characterization of disease, and prognosis is needed.

In recent years, it has been demonstrated that certain substances, including proteins, referred to as biomarkers, are expressed differentially in the diseased tissue and specimens versus the normal tissue and specimens. For example, it is believed that a differentially expressed protein that is found to be present in diseased tissue of many patients, while being absent in the normal tissue, is a candidate biomarker for that disease. Rasmussen et al., Electrophoresis 15:406-416 (1994); Hong Ji et al., Electrophoresis 15:391-405 (1994); Prasad S. C. et al., Int. J. Oncology 14:529-534 (1999); Soldes O. S. et al., British J. of Cancer 79(3/4):595-603 (1999). Biomarkers, hence, provide an additional measure for medical diagnosis and prognosis.

Often, however, a single biomarker may be insufficient for accurate diagnosis of disease onset, and the search continues for the optimal panel of biomarkers that together can provide a profile for a given disease or condition at various stages of its pathology. Emmert-Buck, M. R. et al., Mol. Carcinogenesis 27:158-165 (2000). It is envisioned that a combination of biomarker information, as well as the traditional indicia of medical diagnoses, can provide a more accurate and early detection system.

In some instances, the diagnostic and prognostic problems associated with various diseases and conditions are made more complicated by the fact that not enough biomarkers for these diseases have been found yet. Hence, there is a need in the art to rapidly identify such biomarkers. But even when a panel of biomarkers are known for a given disease or condition, no integrated system is yet available that accurately and expediently detects and analyzes the protein profile of a given patient so that a timely diagnosis, preferably at the onset of the disease or condition, can be made and the needed course of treatment started at an early stage when the disease or condition is more likely to be responsive to treatment.

The above references are incorporated by reference herein where appropriate for appropriate teachings of additional or alternative details, features and/or technical background.

The above references are incorporated by reference herein where appropriate for appropriate teachings of additional or alternative details, features and/or technical background.

### SUMMARY OF THE INVENTION

In view of the above described problems and limitations of the prior art, it is an object of the invention to solve at least the above problems and limitations by providing at least the advantages described hereinafter.

The present invention relates to a database of protein patterns associated with diseases or other biological conditions.

The present invention also relates to a database that stratifies patients having common diagnosis and clinical outcomes.

The present invention also relates to a database that contains patient clinical information, images, mass spectrometer spectra and data analysis.

The present invention also relates to an algorithm for analyzing protein expression data.

The present invention also relates to an artificial neural network for analyzing protein expression data.

The present invention also relates to an algorithm for recognizing informative patterns of protein expression that can be correlated with clinical parameters and manifestations of disease.

The present invention also relates to a system and methodology for creating a comprehensive protein profile.

The present invention also relates to a system and methodology for identifying protein patterns associated with predetermined biological characteristics.

The present invention also relates to a system and methodology for identifying protein patterns associated with predetermined clinical parameters.

The present invention also relates to a system and methodology for identifying protein patterns associated with predetermined medical conditions.

The present invention also relates to a system and methodology for identifying protein patterns associated with predetermined diseases.

The present invention also relates to a system and methodology for predicting the existence or non-existence of at least one predetermined biological characteristic.

The present invention also relates to a system and methodology for predicting the presence of disease in an animal body, such as a mammal.

The present invention also relates to a system and methodology for rapidly identifying proteins associated with disease or other biological conditions that can be used as biomarkers in diagnostic applications.

The present invention also relates to a system and methodology for using a biomarker protein as a non-invasive imaging target for one or more sites of diseased cells in a mammalian body.

The present invention also relates to a system and methodology for using biomarker proteins as a therapeutic target for treatment of disease or other biological conditions.

The present invention also relates to a system and methodology for discovering proteins that are useful as imaging or therapeutic targets of disease.

The present invention also relates to protein biomarkers for monitoring the course of a disease, and for determining appropriate therapeutic intervention.

The present invention also relates to a system and methodology for using biomarker proteins as targets for drug delivery systems in a mammalian body in order to enhance drug efficacy.

The present invention also relates to specific protein biomarkers of various cancers, such as lung cancer (particularly non-small cell lung cancer), colon cancer, breast cancer, prostate cancer, ovarian cancer, lymphoma, melanoma and CNS tumors, including marcrophage migration inhibitory factor (MIF) and cyclophilin A.

The present invention also relates to methods of treating a cancer, such as lung cancer particularly non-small cell lung cancer), colon cancer, breast cancer, prostate cancer, ovarian cancer, lymphoma, melanoma and/or CNS tumors, by administering an agent that affects the expression and/or function(s) of a protein biomarker associated with that cancer, such as marcrophage migration inhibitory factor (MIF) and cyclophilin A.

Additional advantages, objects, and features of the invention will be set forth in part in the description which follows, and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIG. 1 is a block diagram of a cell protein profiling and diagnostic system, in accordance with the present invention;

FIG. 2A is a flowchart of one preferred method of identifying and storing cell protein patterns using the system of FIG. 1, in accordance with the present invention;

FIG. 2B is a flowchart of one preferred diagnosing method using the system of FIG. 1, in accordance with the present invention;

FIG. 2C is a flowchart of one preferred method of preparing a tissue sample for protein fractionation, in accordance with the present invention;

FIG. 3 is a graph showing representative spectra of tumor and normal lung lysates analyzed on a cation exchange surface, in accordance with the present invention;

FIG. 4 is a graph showing representative spectra of tumor and normal lung lysates analyzed on an anion exchange surface;

FIG. 5 is a graph showing representative spectra of tumor and normal lung lysates analyzed on an immobilized metal infinity surface;

FIG. 6 is the nucleic acid sequence of human macrophage migration inhibitory factor [SEQ ID NO. 1] and the deduced amino acid sequence of human MIF [SEQ ID NO. 2]; and

FIG. 7 is the nucleic acid sequence of human cyclophilin A [SEQ ID NO. 3] and the deduced amino acid sequence of human cyclophilin A [SEQ ID NO. 4].

### DETAILED DESCRIPTION OF PREFERRED EMBODIMENTS

I. Systems and Methods of Determining Differential Protein Expression

The present invention provides an apparatus and methodology for rapidly identifying new biomarkers, generating a comprehensive database of biomarkers and other indicia for medical diagnosis and prognosis, generating substantially complete protein profiles for a given population, and allowing generation and comparison of the protein profile of a given individual against the population profile, thereby detecting the differences that point to the presence or absence of disease or other biological conditions.

In a preferred embodiment of the invention, a tissue sample or specimen, such as urine, blood, or other readily obtainable and minimally invasive biological sample, is obtained from the patient. The sample is used to generate cell or specimen lysates. Any methodology, including the ones described herein below, may be used to make cell or specimen lysates. p Next, the total complex protein composition is fractionated into sub-groups. Any methodology may be used to fractionate the proteins into sub-groups, as long as the complexity of the original protein mixture is reduced. Protein fractionation may be done based on any given property, e.g. size, charge, isoelectric point, or hydrophobicity, as long as the fractions obtained are sufficiently reduced in complexity to permit detection by mass spectrometry of the greatest possible proportion of all the proteins in the fraction.

It is advisable to use one or several different types of separation steps in order to fractionate the cell lysates prior to mass spectrometric analysis. Such chromatographic steps include, but are not limited to, the following: normal and reversed-phase high performance liquid chromatography (HPLC), ion-exchange chromatography, size exclusion chromatography, 1D or 2D gel electrophoresis, isoelectric focusing, and capillary electrophoresis. Experimental results have shown that the use of reversed-phase HPLC to fractionate cell lysates can affect the number and distribution of proteins detected by spectrometry. When the eluant from the reversed-phase HPLC separation is subjected to spectrometry (e.g. MALDI) analysis, an increased number of proteins are clearly detected.

The number of fractions generated for analysis may vary based on the given particulars at hand, described below. It is expected, however that the fractions generated would contain as few as less than 10 to as high as 1,500 proteins. In general, HPLC will generate more complex fractions than a gel fractionation method, such as 2D gel electrophoresis. However, since the proportion of fractioned proteins that are analyzable by mass spectrometry will differ depending on the fractionation method used, the most effective method will involve more than one fractionation scheme.

After fractionating the total cell or specimen protein content into sub-groups or fractions, each protein fraction or sub-group is then analyzed by mass spectrometry using, for example, Matrix Assigted Laser Desorption/Ionization (MALDI) or Surface-Enhanced Laser Desorption Ionization (SELDI) time-of-flight mass spectrometry. Without fractionation, mass spectrometry analysis of complex protein mixtures such as those in whole cell lysates can be compromised due to the fact that different peptide and protein analytes can experience preferential desorption/ionization in the mass spectrometry process. In some cases, signal suppression effect can be so severe that certain peptides and proteins are not detected in the presence of others.

In designing the present invention, the initial mass spectrometry experiments of tumor cell lysates were carried out using mass spectrometry samples directly from the cell or specimen lysates without any fractionation step (see Example 1 below). This, however, typically allowed detection on the order of 30-50 peptides and proteins, an estimated less than 1% of the total protein content of the cell. To visualize many more proteins and produce the most comprehensive disease profile possible, the protein fractionation step was devised to be carried out prior to mass spectrometry analysis, so that each fraction will generate a diverse protein spectrum. The fractionation step, which makes use of a variety of separation techniques, increases the number of proteins identified in the complete expression profile of the lysate.

The data output from the mass spectrometry is an array, or spectrum, of peaks with each peak representing a protein or group of proteins present in a given sample. The location of any given peak on the x-axis is related to the molecular mass and charge of the protein, while the height of the peak is associated with the relative abundance of the protein ion. For a given set of experimental conditions, the spectrum represents a molecular profile of the protein sub-group or fraction of the expressed proteins in a given specimen.

By comparing the protein spectra between different specimens or between the specimen and the established control(s), differences between them can be ascertained. For example, by comparing the spectrum of healthy tissue to a spectrum of diseased tissue from the same patient, differences in the expression of specific proteins can be detected. Hence, a differentially expressed protein or proteins that are found in diseased tissue of many patients, while being absent in the normal tissue, is a candidate biomarker for that disease. Similarly, the differences between the protein profile of a given patient and the profile generated from studying a population to which the patient is related, are indicative of the presence or absence of a biomarker, which can assist in the diagnosis and/or prognosis of a disease or biological condition.

The present invention makes use of neural networks and other analysis techniques to determine which proteins are common to patients with the same disease. In addition, the data is mined to determine the differences in protein expression between the diseased/abnormal and normal subjects (and other diseases or abnormalities), and thus create a series of patterns of protein expression unique to that specific disease or biological condition. Individual proteins found in specific diseases or abnormalities, and not found in normal specimens, can be identified as possible therapeutic targets.

This creation of protein patterns for specific diseases or other biological conditions will allow the system described herein to analyze any unknown specimen and determine the diagnosis with prognostic and therapeutic implications.

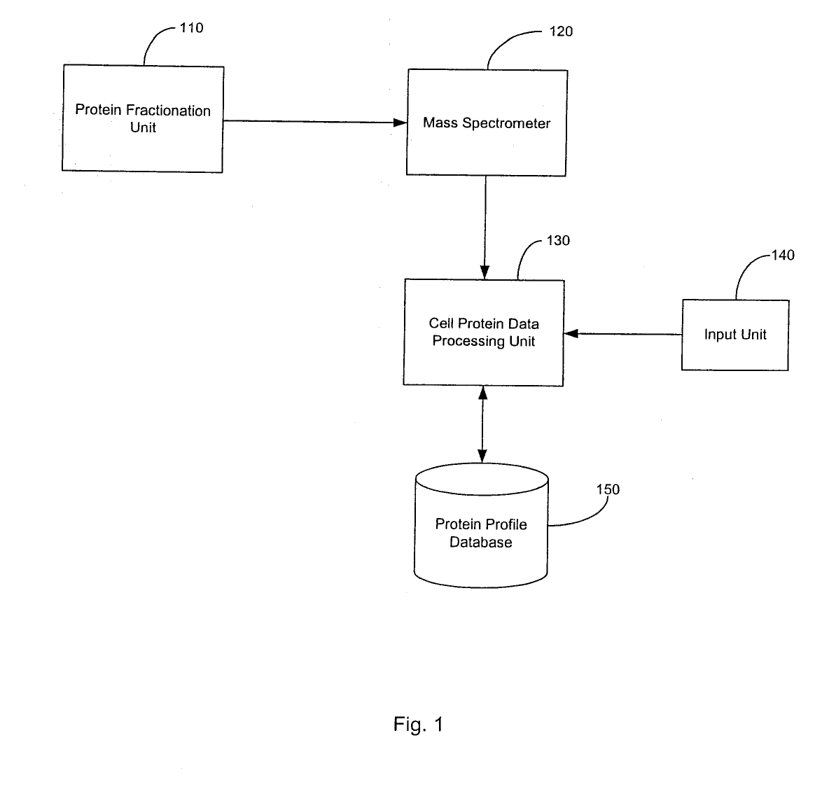

FIG. 1 is a block diagram of a cell or specimen protein profiling and diagnostic system 100, in accordance with the present invention. The system comprises a protein fractionation unit 110, a mass spectrometer 120, a cell protein data processing unit 130, an input unit 140 and a protein profile database 150.

The system 100 is used to create substantially complete protein profiles for samples, identify protein patterns in the cell protein profiles that are associated with subject characteristics, such as biological conditions and diseases, and storing these protein profiles and identified protein patterns for later use in diagnostic applications.

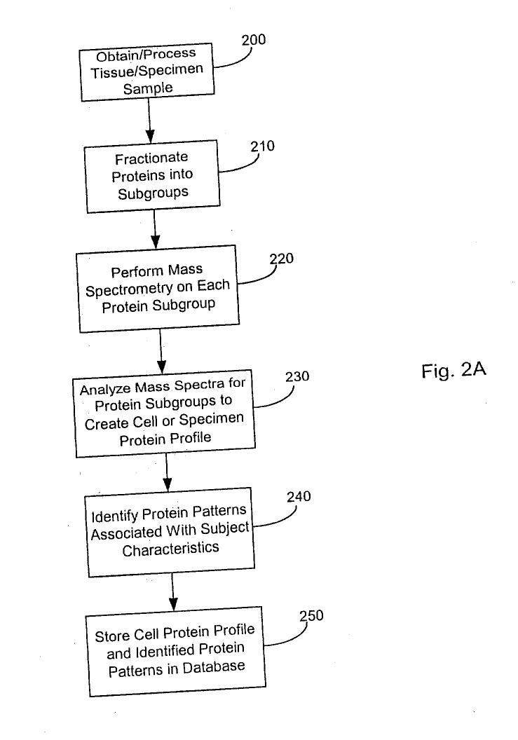

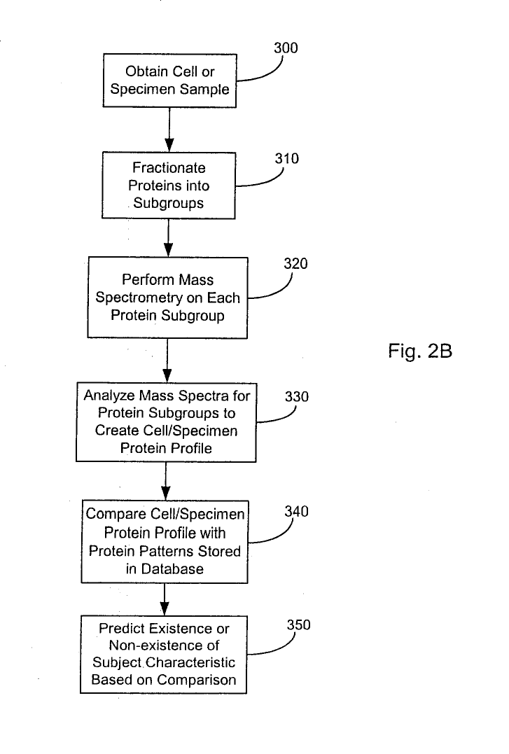

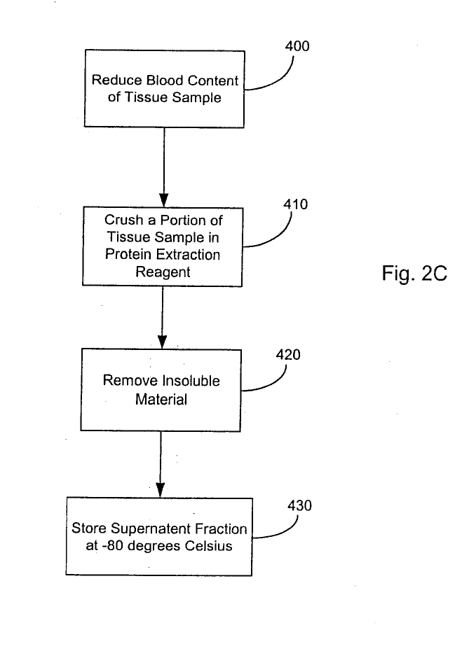

The operation of the system 100 will be further described in connection with FIGS. 2B and 2C, which are flowcharts of a preferred method of identifying and storing disease protein patterns, and a preferred diagnosing method, respectively. The method of FIG. 2B begins at step 200, where a tissue sample is obtained from a subject. The type of tissue sample selected depends on the type of disease protein pattern that one wants to identify. However, the tissue sample is typically not composed of a homogeneous population of one cell type. For example, a specimen of lung tumor is composed of cancer cells, normal lung cells, blood cells, endothelial cells, etc. However, tumor specimens from two different subjects may contain similar populations of cells. This could be ascertained by the examination of stained thin sections of the tissue sample being analyzed.

At step 210, the protein fractionation unit 110 fractionates proteins from the tissue sample into protein subgroups. A tissue sample can contain tens of thousands of different proteins, and possibly over one hundred thousand distinct proteins if post-translational modification is performed. Mass spectrometers currently available do not have the resolution required to visualize every distinct protein in a tissue sample.

Accordingly, one aspect of the present invention is the recognition that fractionating the proteins found in the tissue sample into multiple subgroups, and performing mass spectrometry on each protein subgroup, will increase the number of proteins detected in a given sample.

Any technique can be used by the protein fractionation unit 100 to fractionate the proteins found in the tissue sample into protein subgroups. For example, the fractionation can be done by size, charge, isoelectric point or hydrophobicity. Whatever technique is used, the fractions obtained must be sufficiently reduced in complexity to permit detection, by mass spectrometry, of the largest possible proportion of all the proteins contained in the fraction.

A preferred method for performing the protein fractionation is analytical reversed-phase high performance liquid chromatography (RP-HPLC). One example of an instrument that can be used to perform the analytical RP-HPLC is a Dynamax SD-200 solvent delivery system, and a Dynamax Variable Wavelength UV/Visible Absorbance Detector.

Analytical RP-HPLC is preferably performed on a C4 Vydac column (0.46×15.0 cm, 300 angstroms) at a flow rate of 1 mL-min. Separations are preferably performed using linear gradients of Buffer B in A (Buffer A=0.1% TFA in water, and Buffer B=90% acetonitrile in water containing 0.09% TFA). A 0 to 67% gradient of Buffer B in A is preferably used for the separation. However, other gradient schemes and buffer compositions can also be used.

A fractionation scheme such as analytical RP-HPLC will generate 20 fractions. Thus, assuming 37,000 different proteins are present in the tissue sample, each fraction will have approximately 1,850 proteins.

A gel-base fractionation technique is able to generate more fractions than the analytical RP-HPLC technique. For a 1D gel that is 10 cm long, one can obtain from 100-1,000 fractions, depending on whether the fraction is 1 mm or 0.1 mm in length. The number of fractions increases dramatically with a 2D gel to 10,000-100,000 fractions, depending on the size of the spot analyzed (1.0 or 0.1 mm on a side). Although not all spots will contain protein, one still obtains a large number of fractions.

As discussed above, fractionation will typically be able to generate fractions that contain as few as less than 10 proteins per fraction, to as many as over 1,500 proteins per fraction. In general, analytical RP-HPLC will generate more complex fractions than gel fractionation. However, since the proportion of a fractionated proteins that are analyzable by mass spectrometry will differ depending on the fractionation method used, the most affective protein fractionation method may involve using more than one fractionation technique. Other fractionation techniques that can be used include, but are not limited to, normal HPLC ion-exchange chromatography, size exclusion chromatography, and capillary electrophoresis.

Clearly, to avoid protein degradation, appropriate steps should be taken to preserve the protein content of the samples. The tissue sample should be prepared as soon as possible after it is obtained, or stored in liquid nitrogen or otherwise at approximately −80° C. Once the proteins and the tissue sample are fractionated, the protein fractions should be analyzed, or stored in liquid nitrogen or otherwise at approximately −80° C.

At step 220, mass spectrometry is performed on each protein subgroup that comes out of the fractionation process. The mass spectrometry is preferably performed using Matrix Assisted Laser Desorption/Ionization Time-Of-Flight (NALDI-TOF) mass spectrometry. However, a variety of other mass spectrometric methods such as SELDI and Electrospray Ionization (ESI) may also be used.

Each protein sub-group is preferably prepared for MALDI-TOF mass spectrometry by combining approximately 1 μL of the protein sub-group with approximately 30 μL of MALDI substrate solution (or with solution appropriate for whatever mass spectrometric procedure is used), which contains a saturated aqueous solution of sinapinic acid containing 50% acetonitrile and 0.1% trifluoracetic acid (TFA), or other matrices.

The saturated solution of sinapinic acid is preferably prepared by adding solid sinapinic acid to a 50:50 (v/v) solution of water and acetonitrile with 0.1% (v/v) of TFA. The approximate ratio of (30:1) of MALDI substrate solution to protein lysate extract can be varied beyond this ratio on a case-by-case basis to effect an optimal concentration for MALDI-TOF mass spectrometry for a given situation.

For each protein sub-group that is run through the mass spectrometer 120, a mass/amplitude spectrum is generated. Specifically, the time-of-flight data for a given protein in a mixture is translated into the mass/charge ratio for the protein, or m/z. Because the charge is typically assumed to be +1, the m/z values in a spectrum are considered to be equivalent to the molecular mass of the protein plus the mass of a proton (i.e., 1). The resulting data is in the form of a X-Y plot where peaks, representing individual proteins or groups of proteins, are arrayed along the x-axis at their respective m/z values. The height of each peak is proportional to the detector response and, hence, can be interpreted as the relative abundance of the protein ions contributing to the peak.

At steps 230 and 240, the cell protein data processing unit 120 analyzes the mass spectra for each of the protein sub-groups to create a cell protein profile, and identifies protein patterns associated with subject characteristics. Subject characteristics typically include patient clinical information such as age, sex, disease, outcome, stage at presentation and response to therapy.

The subject characteristics are input to the cell protein data processing unit 130 with input unit 140. Input unit 140 is suitably a computer that stores subject information.

The cell protein data processing unit 130 obtains information regarding protein expression patterns that are specific to diseases by comparing the mass spectrometer spectra between specimens representing diseased and healthy states. The cell protein profiles and protein patterns identified by the cell protein data processing unit 130 are stored, at step 250, in the protein profile database 150. The database 150 preferably incorporates fields for entry of spectra and for seamless integration of data analysis. Each database entry preferably contains patient clinical information, images (CT, PET radiographs), mass spectrometer spectra, and data analysis.

FIG. 2B is a flowchart of one preferred diagnosing method, utilizing the system 100 of FIG. 1. Steps 300-330 are similar to steps 200-230 in the method of FIG. 2A, and thus will not be explained again.

At step 340, the cell protein data processing unit compares the cell protein profile with the protein patterns previously identified and stored in the database 150. At step 350, the existence or non-existence of subject characteristics, such as biological conditions or diseases, are predicted by the cell protein data processing unit 130.

The raw time-of-flight versus amplitude data received by the cell protein data processing unit 130 may consist of tens of thousands of individual measurements for each tissue sample analyzed. While it may be possible to obtain useful information regarding protein expression differences among very small groups of tissue samples with the naked eye, a through comparison among many hundreds of tissue samples is preferably performed with a computer algorithm that is executed by the cell protein profiling unit 130.

Accordingly, the cell protein data processing unit 130 preferably utilizes an algorithm to identify the protein patterns associated with subject characteristics, such as predetermined medical conditions or diseases. The algorithm is preferably designed to recognize informative patterns of protein expression that may be correlated with clinical parameters and manifestations of disease. The algorithm is also preferably designed to identify proteins associated with disease that may be used as biomarkers in in vitro diagnostic applications, or as targets for non-invasive imaging or to guide the delivery of cytotoxic or therapeutic agents.

The algorithm may be based on an Artificial Neural Network (ANN). Given N cases, the ANN is preferably trained on N-1 cases, and then validated on the one case left out. This process is preferably repeated N times until each case has served as a validation case, and then all N results are combined. The resulting ANN analyzes each peak separately and attempts to predict if it originated from a diseased tissue sample or a normal tissue sample.

When an ANN, as described above, was used on a data set with a total 248 peaks, a 93% sensitivity and a 61% specificity in identifying spectra as “disease” or “normal” was achieved. The sensitivity can be increased to approximately 95% by combining the original ANN with a second ANN based on a different molecular mass range. However, this additional classification step decreases the specificity to 58%.

A second preferred algorithm uses all data points contained in a mass spectrometer spectrum, as opposed to using only the peaks identified by the mass spectrometer software. With this algorithm, the data are first filtered in order to produce a uniform base line amount among all sample spectra. Next, the sample data sets are put through a T-squared test to determine which bins are the most valuable in terms of their ability to separate the two sample sets (diseased and normal) of data.

The test yields a P-value for each bin, which reflects the probability that the means of the two groups of data in that bin are equal. A very low P-value indicates that the two means are not close to each other, and thus that bin has a reasonable capability of separating the sample sets. The lower the P-value, the more separable the data is in that particular bin.

FIG. 2C is a flowchart of a preferred method for preparing the tissue sample for protein fractionation, as part of steps 210 and 310 in the methods of FIGS. 2A and 2B, respectively. The method begins at step 400, were the blood content of the tissue sample is reduced by incubating the tissue sample in 10 mL PBS at approximately 4° C. for approximately 30 minutes.

Then, at step 410, a portion of the tissue sample is crushed in a protein extraction reagent. Specifically, a small portion of the cell sample (preferably 10-20 mg wet weight) is preferably placed into a 1.5 ml mictocentrifuge tube containing 65 μL Mammalian Protein Extraction Reagent (M-PER). The portion of the tissue sample is crushed in the M-PER preferably using a plastic microcentrifuge-sized pestle, and then shaken for approximately 10 minutes at approximately 40° C.

Next, at step 420, insoluble material is removed by centrifugation at 16,000×g at approximately 4° C. for approximately 20 minutes. At step 430, the supernatant fraction is stored, preferably in a clean microcentrifuge tube, in liquid nitrogen or otherwise at approximately −80° C. until it is used.

II. Protein Biomarkers and Therapeutic and Diagnostic Uses Thereof

Other preferred embodiments of the present invention are directed to specific protein biomarkers identified using the methods and systems described above, as well as therapeutic and/or diagnostic applications thereof.

Particularly preferred embodiments of the present invention relate to specific protein biomarkers that are associated with cancer and similar conditions involving cell proliferation and/or differentiation. Among these embodiments are therapeutic methods involving agents that affect the function and/or expression of one of more such protein biomarkers, as well as diagnostic methods involving the measurement of expression of one or more such protein biomarkers.

Specific protein biomarkers that have been identified using the methods and systems described above include protein biomarkers for various cancers, such as lung cancer (particularly non-small cell lung cancer), colon cancer, breast cancer, prostate cancer, ovarian cancer, lymphoma, melanoma and CNS tumors. Such protein biomarkers include Macrophage Migration Inhibitory Factor (MIF) and Cyclophilin A (CyP-A), both of which have been identified using using the methods and systems of the present invention as protein biomarkers for the various cancers listed above.

Accordingly, particularly preferred embodiments of the present invention include methods of diagnosing cancer, such as lung cancer (particularly non-small cell lung cancer), colon cancer, breast cancer, prostate cancer, ovarian cancer, lymphoma, melanoma and/or CNS tumors, by detecting the level of protein biomarkers such as MIF and CyP-A. Such methods preferably include detecting the level of a protein biomarker such as MIF and CyP-A per se, either directy (e.g by determining or estimating absolute protein level(s) of MIF and/or CyP-A in a sample obtained from a patient, either a body fluid generally, such as blood or lymph, or in a particular tissue, such as lung tissue) or indirectly (e.g. by comparing to the level of MIF and/or CyP-A in a second sample) and detecting the level of expression of the nucleic acid sequence(s) encoding the protein biomarker(s), either directly (e.g by determining or estimating absolute mRNA level(s) in a sample obtained from a patient) or indirectly (e.g. by comparing to the mRNA level in a second sample).

According to particularly preferred embodiments of the present invention, the level of MIF and/or CyP-A present in a sample of tissue obtained from a patient is compared to a standard level. Such a standard level may be obtained from a sample of similar tissue from a patient known to have the suspected cancer, e.g. non-small cell lung cancer, or from a patient known not to have the suspected cancer. Most preferably, the level of MIF and/or CyP-A present in a given sample of tissue obtained from a patient is compared to the level of MIF and/or CyP-A present in a sample of normal tissue obtained from the same patient.

In one embodiment of the invention, the diagnostic methods may be performed using a diagnostic agent, such as an antibody or nucleic acid sequence, attached to a solid support. For example, nucleic acid sequence(s) which selectively hybridize to MIF and/or CyP-A may be attached to a “gene chip” as described in U.S. Pat. Nos. 5,837,832; 5,874,219; and 5,856,174. Similarly, an antibody to MIF and/or CyP-A, or fragment thereof, may be attached, either directly or through a linker, to a solid support according to the methods known to those skilled in the art.

Most preferably, the level of MIF and/or CyP-A present in a given sample obtained from a patient is determined using the preferred systems and methods for determining differential protein expression described above, i.e., using processes involving fractionation and mass spectrometry.

Still other preferred embodiments of the present invention include methods of using antibodies to MIF or CyP-A, or fragments thereof, alone or conjugated to a diagnostic agent. Antibodies to MIF and Cyp-A are known and available to those skilled in the art; see, for example, U.S. Pat. Nos. 5,047,512 and 6,080,407.

These antibodies can be used diagnostically to, for example, monitor the development or progression of a tumor as part of a clinical testing procedure to, e.g., determine the efficacy of a given treatment regimen. Detection may be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions.

The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

Antibodies for MIF and CyP-A may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., N.Y.). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sephatose beads). For further discussion regarding immunoprecipitation protocols see, e.g., Ausubel et al, eds, 1994, Current Protocols in Molecular Biology, Vol. 1, John Wiley & Sons, Inc., N.Y. at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., N.Y. at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., N.Y. at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

In addition to the above, still other particularly preferred embodiments of the present invention include methods of treating various cancers, lung cancer (particularly non-small cell lung cancer), colon cancer, breast cancer, prostate cancer, ovarian cancer, lymphoma, melanoma and/or CNS tumors, by administering to a patient in need thereof an effective amount of at least one agent that affects the function and/or expression of at least one protein biomarker for that particular cancer, such a MIF or CyP-A in the case of the various cancers listed above. Such agents include antibodies that bind to, and thereby affect the function of, these protein biomarkers.

Still other agents anti-sense constructs prepared using antisense technology. Anti-sense technology can be used to control gene expression through triple-helix formation or anti-sense DNA or RNA, both of which are based on binding of a polynucleotide to DNA or RNA. For example, the 5′ coding portion of the nucleotide sequences which encode for MIF or CyP-A may be used to design an anti-sense RNA oligonucleotide of from about 10 to 40 base pairs in length. A DNA oligonucleotide is designed to be complementary to a region of the gene involved in transcription (triple-helix, see Lee et al., Nucl. Acids Res., 6:3073 (1979); Cooney et al, Science, 241:456 (1988); and Dervan et al, Science, 251:1360 (1991)), thereby preventing transcription and the production of MIF and/or CyP-A. The anti-sense RNA oligonucleotide hybridizes to the mRNA in vivo and blocks translation of the mRNA molecule into the polypeptides (Okano, J. Neurochem., 56:560 (1991); Oligodeoxynucleotides as Antisense Inhibitors of Gene Expression, CRC Press, Boca Raton, Fla. (1988)). The oligonucleotides described above can also be delivered to cells such that the anti-sense RNA or DNA may be expressed in vivo to inhibit production of MIF and/or CyP-A.

Still other agents include small molecules that bind to or interact with MIF and/or CyP-A and thereby affect the function thereof, such as an agonist or antagonist of at least one bioactivity of MIF and/or CyP-A, and small molecules that bind to or interact with nucleic acid sequences encoding MIF and/or CyP-A, and thereby affect the expression of these protein biomarkers.

Because the conjugates of the present invention can be used for modifying a given biological response, the therapeutic agent is not to be construed as limited to classical chemical therapeutic agents. For example, the therapeutic agent may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator, an apoptotic agent, e.g., TNF-alpha, TNF-beta, AIM I (See, International Publication No. WO 97/33899), AIM II (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are known and available to those skilled in the art (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al, “Antibodies For Drug Delivery”, in Controlled Drug Delivery (2nd Ed.), Robinson et al (eds.), pp. 623-53 (Marcel Dekker, Inc. 1987); Thorpe, “Antibody Carriers Of Cytotoxic Agents In Cancer Therapy: A Review”, in Monoclonal Antibodies ′84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol Rev. 62:119-58 (1982)). Alternatively, an antibody can be conjugated to a second antibody to form an antibody heteroconjugate as described by Segal in U.S. Pat. No. 4,676,980.

An antibody to MIF and/or CyP-A, or a fragment thereof, administered alone or in combination with cytotoxic factor(s) and/or cytokine(s), can be used as a therapeutic agent.

Such an antibody or fragment thereof may also be conjugated to an active moiety such as a cytotoxin, e.g., a cytostatic or cytocidal agent, a drug or a radioactive metal ion, e.g., alpha-emitters such as, for example, ²¹³Bi. As used herein, the term “cytotoxin” includes any agent that is detrimental to cells. Examples of suitable cytotoxins include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Illustrative examples of suitable drugs include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DD) cisplatin), anthracyclines (e.g., daunorubicin (daunomycin) and doxorubicin), antibiotics (e.g., dactinomycin (actinomycin), bleomycin, mithramycin, and anthramycin (AMC)), and anti-mitotic agents (e.g., vincristine and vinblastine).

The present invention is further directed to antibody-based therapies which involve administering antibodies to MIF and/or CyP-A to a patient for treating cancer, lung cancer (particularly non-small cell lung cancer), colon cancer, breast cancer, prostate cancer, ovarian cancer, lymphoma, melanoma and/or CNS tumors. A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

Antibodies to MIF anbd/or CyP-A may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

These may be administered alone or in combination with other types of treatments known and available to those skilled in the art for treating cancers, lung cancer (particularly non-small cell lung cancer), colon cancer, breast cancer, prostate cancer, ovarian cancer, lymphoma, melanoma and/or CNS tumors (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against protein biomarkers such as MIF and or CyP-A, fragments or regions thereof, for both immunoassays directed to and therapy of disorders involving abnormal cell differentiation and/or proliferation, such as lung cancer (particularly non-small cell lung cancer), colon cancer, breast cancer, prostate cancer, ovarian cancer, lymphoma, melanoma and/or CNS tumors. Such antibodies, fragments, or regions, will preferably have an affinity for protein biomarkers such as MIF and or CyP-A, including fragments thereof.

Accordingly, the present invention also involves methods of treating a cancer, such as lung cancer (particularly non-small cell lung cancer), colon cancer, breast cancer, prostate cancer, ovarian cancer, lymphoma, melanoma and/or CNS tumors, by administering to a patient in need thereof a therapeutically effective amount of at least one agent that affects the expression or function of MIF and/or CyP-A. Such an agent may be administered alone or in a pharmaceutical composition.

Small molecules which inhibit at least one bioactivity of a protein biomarker such as MIF are known and available to those skilled in the art; see, e.g., Dios et al. J. Med. Chem. 45:2410-2416 (2002). Such molecules include imine conjugates prepared by coupling amino acids, particularly aromatic amino acids, with benzaldehyde derivatives.

Formulations and methods of administration that can be employed when the agent comprises a nucleic acid or an immunoglobulin are described above; additional appropriate formulations and routes of administration, e.g. for small molecules, can be selected from among those described herein below.

Various delivery systems are known and can be used to administer a therapeutic compound, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In addition, pulmonary administration can also be employed, e.g., by use of an inhaler or nebulizer, and formulation with an aerosolizing agent.

In a specific embodiment, it may be desirable to administer these pharmaceutical compositions locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the pharmaceutical composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327).

In yet another embodiment, the pharmaceutical composition can be delivered in a controlled release system. In one embodiment, a pump may be used (see Langer, supra; Sefton, CRC Crit. Ref Biomed. Eng. 14:201 (1987); Buchwald et al., Surgery 88:507 (1980); Saudek et al., N. Engl. J. Med. 321:574 (1989)). In still another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J.Neurosurg. 71:105 (1989)). In yet another embodiment, a controlled release system can be placed in proximity of the therapeutic target, i.e., the lung in the case of non-small cell lung cancer, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

In a specific embodiment where the therapeutic agent is a nucleic acid, such as an anti-sense nucleic acid, the nucleic acid can be administered in vivo to inhibit expression of its target protein, such as MIF or CyP-A, or by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

In any of the therapeutic methods of the present invention, pharmaceutical composition(s) employed generally comprise a therapeutically effective amount of a therapeutic agent, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered.

Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin.

Such compositions will contain a therapeutically effective amount of the active agent, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the therapeutic agent which will be effective in the treatment, inhibition and prevention of a cancer, lung cancer particularly non-small cell lung cancer), colon cancer, breast cancer, prostate cancer, ovarian cancer, lymphoma, melanoma and/or CNS tumors, can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the lung) of the antibodies by modifications such as, for example, lipidation.

Still other preferred embodiments of the present invention include methods of screening compounds to identify therapeutic agents for diseases involving abnormal cell proliferation and/or differentiation, such as lung cancer (particularly non-small cell lung cancer), colon cancer, breast cancer, prostate cancer, ovarian cancer, lymphoma, melanoma and CNS tumors. For example, potential therapeutic agents may be identified by screening for the ability to inhibit at least one bioactivity of MIF, such as MIF tautomerase activity or MIF pro-inflammatory activity, using methods and techniques known and available to those skilled in the art; see, for example, Dios et al, J. Med. Chem. 45:2410 (2002) and U.S. Pat. No. 6,080,407. Similarly, other potential therapeutic agents for cancer(s) may be identified by screening for the ability to inhibit at least one bioactivity of CyP-A, such as CyP-A immunosupressant activity, using methods and techniques known and available to those skilled in the art; see, for example, U.S. Pat. No. 5,047,512.

### EXAMPLES

The following examples are intended to further illustrate certain embodiments of the invention and are not intended to be limiting in nature.

### Example 1

MALDI samples of tumor and normal cell lysates were prepared by combining 1 μl of the unpurified cell lysate with 30 μl of a saturated aqueous solution of sinapinic acid containing 50% acetonitrile and 0.1% trifluoracetic acid (TFA). Ultimately, 1-2 μl of the resulting mixture was deposited on the MALDI sample stage, and the solvent was evaporated at room temperature. MALDI mass spectra were acquired on a Voyager DE Biospectrometry Workstation (PerSeptive Biosystems, Inc., Framingham, Mass.) in the linear mode using a nitrogen laser (337 nm).

All mass spectra were collected in the positive-ion mode, and the spectra represent the sum of approximately 32 laser shots. The raw intensity versus time data was smoothed using a Savitsky-Golay smoothing routine prior to mass calibration using an internal standard. Using the simple MALDI sample preparation described above, approximately 30-50 peptides and proteins were detected, which is less than 1% of the total protein content of the cell. Interestingly, in this relatively small population of proteins, at least 1 protein was identified that appears unique to tumor cell lysates. These profiles can be used to accurately separate tumor from normal samples and other diseases based on their protein spectrum.

### Example 2

One of the differences between SELDI and conventional MALDI-TOF is the ProteinChip™ technology for sample application. ProteinChips are available with a variety of chemical surfaces, which permits the capture and analysis of whole classes of proteins based on their charge, hydrophobicity, or metal binding capablity. The analysis of a biological specimen using just one surface may give information on 40-60 different proteins. By using a series of different surfaces and different wash conditions, it is possible to differentiate 500-1,000 proteins. However, sample preparation and analysis must be optimized for each ProteinChip surface and for each sample type.

ProteinChip surfaces include cation exchange, anion exhange, reverse phase, and imobilized metal affinity capture. Protocals for binding sample to the surfaces and subsequent wash steps are developed much the same way as for column chromotography employing equivalent separation matrices. For example, initial studies using the cation exchange surface have been in a low pH buffer in order to maximize the number of proteins adsorbed to the surface. Potential disease-specific biomarkers identified in the screens can then be partially purified on the ProteinChip surface using wash buffers of progressively higher pH.

FIG. 3 shows representive spectra of tumor (top) and normal (bottom) lung lysates analyzed on a cation exchange surface (WCX-2). The numbers associated with the peaks are mass/charge (m/z) values. Since the charge is +1, the values represent the molecular mass of each protein. The large peak at 22600 Da and the tumor lysate is absent in a normal lung tissue. Likewise, there are peaks at approximately 28,000 and 31,000 Da that present in the normal, but not the tumor. Following verification of these protein expression differences using several different tumor/normal tissue pairs, one can began to isolate these proteins on the chip surface. Since the molecular masses determined by SELDI are very accurate, protein identity can often be achieved by simply searching web-based databases using the molecular mass value. If this is unsuccessful, the isolated protein can be digested with a protease and the resultant peptides separated on the SELDI and peptide fingerprint databases searched.

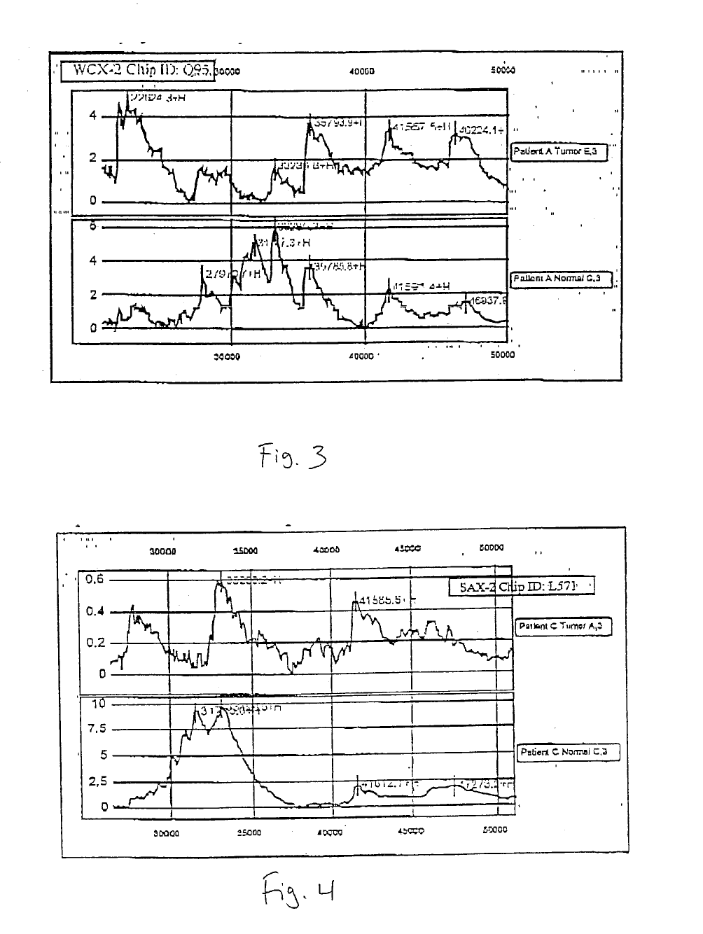

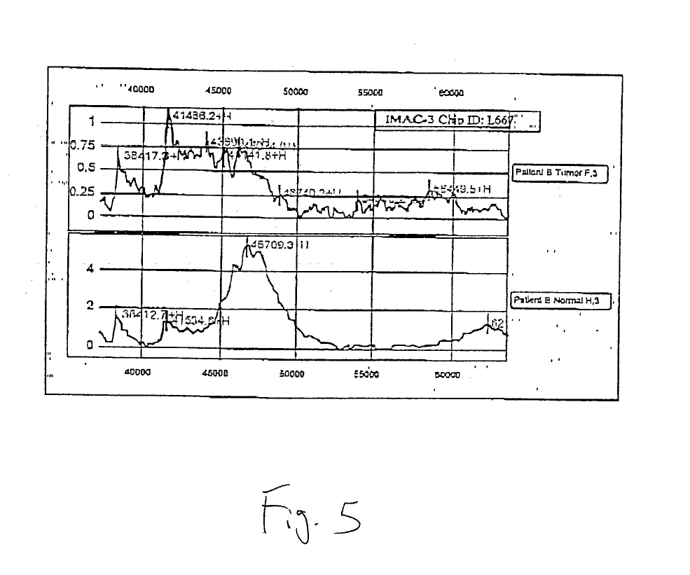

In addition to protocols for the cation exchange surface, protocols for anion exchange (SAX-2) and imobilized metal infinity (IMAC-3) have been derived. Representative spectra from each are shown in FIGS. 4 and 5, respectively.

It is evident that each ProteinChip surface captures a different set of proteins, and each set displays tumor/normal protein expression differences. In order to survey the largest possible set of expressed proteins, all specimens are prefably analyzed using multiple ProteinChip surfaces.

Having now fully described this invention, it will be understood to those of ordinary skill in the art that the methods of the present invention can be carried out with a wide and equivalent range of conditions, formulations, and other parameters without departing from the scope of the invention or any embodiments thereof

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

The foregoing embodiments and advantages are merely exemplary and are not to be construed as limiting the present invention. The present teachings can be readily applied to other types of apparatuses. The description of the present invention is intended to be illustrative, and not to limit the scope of the claims. Many alternatives, modifications, and variations will be apparent to those skilled in the art.

The foregoing embodiments and advantages are merely exemplary and are not to be construed as limiting the present invention. The present teaching can be readily applied to other types of apparatuses. The description of the present invention is intended to be illustrative, and not to limit the scope of the claims. Many alternatives, modifications, and variations will be apparent to those skilled in the art. In the claims, means-plus-function clauses are intended to cover the structures described herein as performing the recited function and not only structural equivalents but also equivalent structures.

What is claimed is:

1. A protein profiling system, comprising:
a protein fractionation unit that separates a protein content of a tissue or specimen sample from a respective subject into protein subgroups;
a mass spectrometer that independently performs mass spectroscopy on each of the protein subgroups from the respective subject's sample, and outputs respective mass spectra subgroup data;
a protein data processing unit that analyzes the mass spectra subgroup data to create a protein profile for the tissue or specimen sample, and identifies protein patterns associated with subject characteristics based on the protein profile and information received on the respective subjects; and
a database that stores the protein profile and the identified protein patterns.

2. The system of claim 1, wherein the subject characteristics comprise predetermined biological conditions.

3. The system of claim 2, wherein at least one of the predetermined biological conditions comprises a predetermined disease.

4. The system of claim 1, wherein the protein data processing unit identifies the protein patterns associated with subject characteristics by comparing protein profiles from a plurality of subjects having a common subject characteristic.

5. The system of claim 1, wherein the protein data processing unit uses a neural network to identify the protein patterns associated with subject characteristics.

6. The system of claim 1, wherein the protein data processing unit uses a peak analysis techniques to identify the protein patterns associated with subject characteristics.

7. A diagnostic system, comprising:
a database that stores protein patterns associated with subject characteristics;
a protein data processing unit that separates a protein content of a tissue or specimen sample from a respective subject into protein subgroups;
a mass spectrometer that independently performs mass spectroscopy on each of the protein subgroups from the respective subject's sample, and outputs respective mass spectra subgroup data; and
a diagnostic unit that analyzes the mass spectra subgroup data to create a protein profile for the tissue or specimen sample, and that compares the protein profile with the stored protein patterns to predict the existence or non-existence of at least one subject characteristic in the respective subject.

8. The system of claim 7, wherein the at least one subject characteristic comprises a predetermined biological condition.

9. The system of claim 8, wherein the predetermined biological condition comprises a disease.

10. A biomarker diagnostic method, comprising the steps of:
collecting a tissue or specimen sample;
fractioning protein content from the sample into protein subgroups;
separately performing mass spectroscopy on each of said protein subgroups and storing resulting mass spectra subgroup data;
analyzing said resulting mass spectra subgroup data to yield a protein profile for said sample.

11. The method of claim 10, wherein said protein profile comprises a comprehensive protein profile.

12. The method of claim 10, wherein said analyzing step comprises analyzing said resulting mass spectra subgroup data using an artificial neural network.

13. The method of claim 10, wherein said separately performing step comprises collecting data points corresponding to said mass spectra subgroup.

14. The method of claim 10, wherein said analyzing step comprises determining data points which yield useful diagnostic information.

15. The method of claim 10, wherein said separately performing step comprises collecting data points corresponding to said mass spectra subgroup, and said analyzing step comprises determining data points which yield useful diagnostic information.

16. The method of claim 15, wherein said data points include data points other than peaks of said mass spectra subgroup.

17. A method for rapidly identifying protein biomarkets, comprising the steps of:
collecting a diseased tissue or specimen sample from at least one patient;
fractionating protein content from said diseased tissue or specimen sample into protein subgroups;
separately performing mass spectroscopy on each of said protein subgroups and storing resulting mass subgroup data;
analyzing said resulting mass spectra subgroup data to yield a protein profile for said diseased tissue or specimen sample;
comparing said protein profile for said diseased tissue sample or specimen against at least one protein profile from at least one normal tissue sample or specimen from said patient or other individuals; and
identifying the differences between said diseased tissue sample or specimen and said at least one protein profile for a normal tissue sample or specimen, thereby identifying protein biomarkers.

18. A protein biomarker identified by the method of claim 17.

19. A diagnostic method, comprising:
collecting a tissue or specimen sample from a patient;
fractionating protein content from said sample into protein subgroups;
separately performing mass spectroscopy on each of said protein subgroups and storing resulting mass subgroup data;
analyzing said resulting mass spectra subgroup data to yield a protein profile for said sample;
comparing said protein profile for said tissue sample or specimen against a protein profile library; and diagnosing presence or absence of a disease or other biological condition.

20. A method for treating a disease involving abnormal cell proliferation and/or differentiation, comprising administering to a patient in need thereof a therapeutically effective amount of at least one agent which affects the function and/or expression of at least one protein biomarker associated with said disease.

21. The method according to claim 20, wherein said protein biomarker is Macrophage Migration Inhibitory Factor (MIF) or Cyclophilin-A (CyP-A).

22. The method according to claim 20, wherein said disease is a cancer.

23. The method according to claim 22, wherein said cancer is selected from the group consisting of lung cancer, colon cancer, breast cancer, prostate cancer, ovarian cancer, lymphoma, melanoma and CNS tumors

24. A method for diagnosing a disease involving abnormal cell proliferation and/or differentiation, comprising:
collecting a tissue or specimen sample from a patient;
determining the level of Macrophage Migration Inhibitory Factor (MIF) and/or Cyclophilin-A (CyP-A) in said sample;
comparing the level in said sample with a reference level.

25. The method according to claim 24, wherein said disease is a cancer.

26. The method according to claim 25, wherein said cancer is selected from the group consisting of lung cancer, colon cancer, breast cancer, prostate cancer, ovarian cancer, lymphoma, melanoma and CNS tumors.
